(19) United States
(12) Patent Application Publication
(10) Pub. No.: US 2005/0043620 A1
(43) Pub. Date: Feb. 24, 2005

(54) Diagnostic medical ultrasound system communication network architecture and method

(75) Inventors: Steven G. Fallows, Hatfield, PA (US); Anthony P. Lannutti, Norristown, PA (US)

Correspondence Address: Siemens Corporation;Intellectual Property Department, 170 Wood Avenue South, Iselin, NJ, 08830, US

(73) Assignee: Siemens Medical Solutions USA, Inc.

(21) Appl. No.: 10/644,532

(22) Filed: Aug. 20, 2003

(51) Int. Cl. A61B 8/00; G06F 7/00; G06F 17/30

(52) U.S. Cl. 600/437; 235/375; 707/10

(57) ABSTRACT

A network architecture for facilitating communications among multiple diagnostic medical imaging devices coupled with a network, such as a local area or wide area network, is disclosed. The architecture features communications components included within each device which permit each device to identify itself to all other devices on the network and respond to the identification by another device. This automated identification and recognition process allows each device to effectively automatically discover all of the other devices on the network, as well as automatically self-adjust to accommodate devices as they are added or removed from the network. Once each device has identified other devices on the network that are available for communications, the architecture facilitates the exchange of data between any of the identified devices at the direction of the user.

### BACKGROUND

In hospitals, clinics and other medical settings, it is typical to have multiple diagnostic medical imaging devices available for use. Such diagnostic medical imaging devices include image acquisition devices, such as diagnostic medical ultrasound systems, magnetic resonance imaging systems, computed tomography systems, x-ray systems, etc. Diagnostic medical imaging devices further include image/study review workstations, output devices, such as printers, and image data storage servers. To increase their effectiveness and efficiency, the diagnostic medical imaging devices in a particular hospital, clinic, etc. are typically coupled together over a local or wide area network. The network permits the devices to communicate with each other and share data, for example allowing a user to send image/study data from an image acquisition device to an image reviewing workstation.

Such networks, however, typically require manual configuration which is often complex. To communicate with a particular device, each device on the network must be manually configured to “know” of the other device, such as by programming each device with the IP or MAC address of the device to be communicated with. Further, when devices are added to, or removed from, the network, the other devices on the network must be manually reconfigured accordingly. One solution to manually configuring the network is to provide a central server on the network which manages intra-device communication. All communications are then transmitted via the server to their ultimate destination. However, while this alleviates the need to configure each device on the network to “know” of the other devices on the network, each device must still be configured to communicate with the server and the server must still be configured to “know” all of the devices on the network. In addition, a server is an additional device on the network necessitating additional management resources, increasing overall system complexity and adding an additional point of failure. Further, such indirect communications reduces the efficiency and bandwidth of the overall network. In addition, with either the decentralized or centralized communication schemes described above, adding or removing devices from the network still requires some measure of manual re-configuration.

Accordingly, there is a need for an intra-device communications network for a network of diagnostic medical imaging devices which permits direct “peer to peer”, i.e. decentralized, communications without requiring manual configuration of source and destination devices or manual re-configuration when the network topology or devices present on the network are altered.

### SUMMARY

The present invention is defined by the following claims, and nothing in this section should be taken as a limitation on those claims. By way of introduction, the preferred embodiments described below relate to a communications interface for a first diagnostic medical imaging device, the communications interface operative to couple the first diagnostic medical imaging device to a network. The communications interface includes identification logic operative to periodically identify, via the network, the first diagnostic medical imaging device to other diagnostic medical imaging devices coupled with the network and receive a response therefrom, the identification logic being further operative to recognize other diagnostic medical imaging devices which identify themselves to the first diagnostic medical imaging device. The communications interface further includes configuration logic coupled with the identification logic and operative to automatically configure the first diagnostic medical imaging device to communicate with the other diagnostic medical imaging devices which at least one of respond and identify themselves and communication logic coupled with the identification logic and the configuration logic and operative to facilitate communication of data between the first diagnostic medical imaging device and the other diagnostic medical imaging devices which at least one of respond and identify themselves.

The preferred embodiments further relate to a method for communicating among a plurality of diagnostic medical imaging devices coupled with a network. In one embodiment, the method includes: identifying, automatically by a first device of the plurality of diagnostic medical imaging devices, a second device of the plurality of diagnostic medical imaging devices available for communication via the network; configuring, automatically, the first device to communicate substantially directly with the second device via the network; and facilitating substantially direct communication of data between the first and second devices.

Further aspects and advantages of the invention are discussed below in conjunction with the preferred embodiments.

### BRIEF DESCRIPTION OF THE DRAWINGS

FIG. 1 depicts a block diagram of an exemplary network of diagnostic medical imaging devices for use with the disclosed embodiments.

FIG. 2 depicts a block diagram of an exemplary diagnostic medical ultrasound system for use with the disclosed embodiments.

FIG. 3 depicts a block diagram of an exemplary diagnostic medical imaging review workstation for use with the disclosed embodiments.

FIG. 4 depicts flow charts of the communications configuration processes executed by each of the devices of FIG. 1, according to one embodiment.

FIG. 5 depicts a flow chart of detailing the file transfer process as executed by a device of FIG. 1, according to one embodiment.

### DETAILED DESCRIPTION OF THE DRAWINGS AND PRESENTLY PREFERRED EMBODIMENTS

A network architecture for facilitating communications among multiple diagnostic medical imaging devices coupled with a network, such as a local area or wide area network, is disclosed. Herein, the phrase “coupled with” is defined to mean directly connected to or indirectly connected through one or more intermediate components. Such intermediate components may include both hardware and software based components. The architecture features communications components included within each device which permit each device to identify itself to all other devices on the network, allow identification by the other devices, as well as maintain the present status, i.e. availability, of other devices. This automated identification and recognition process allows each device to effectively automatically discover all of the other devices on the network, as well as automatically self-adjust to accommodate devices as they are added or removed from the network. Once each device has identified other devices on the network that are available for communications, the architecture facilitates the exchange of data between any of the identified devices at the direction of the user.

In one embodiment, the architecture includes the CypressLink communications architecture manufactured by Siemens Medical Solutions USA, Inc., located in Malvern, Pa., which is used to link, via the network, Cypress Imaging Systems and Cypress Review Stations. Cypress Imaging Systems include Cypress hardware units running the Cypress application software, manufactured by Siemens Medical Solutions USA, Inc., located in Malvern, Pa., capable of capturing images and creating patient study files. Exemplary Cypress Imaging Systems include diagnostic medical imaging systems such as the Cypress Echocardiography System manufactured by Siemens Medical Solutions USA, Inc., located in Malvern, Pa. Cypress Review Stations include standard Microsoft Windows based personal computers executing the Cypress Viewer application software, manufactured by Siemens Medical Solutions USA, Inc., located in Malvern, Pa., capable of viewing patient study files. An Image Creation server is any CypressLink capable device that can create Cypress format patient study files, such as a Cypress Imaging system or other imaging system, such as the Sonoline Antares™ Ultrasound Platform manufactured by Siemens Medical Solutions USA, Inc., located in Malvern, Pa., including the appropriate CypressLink components. Cypress is a proprietary format for diagnostic medical imaging data promulgated by Siemens Medical Solutions USA, Inc., located in Malvern, Pa. A Patient Database server includes any CypressLink capable machine that can send/store/receive Cypress format patient study files., such as a Cypress Imaging System or Cypress Review Station.

The disclosed architecture provides the ability for both Cypress Imaging System units and Cypress Review Station units to recognize each other on a local area network (“LAN”) and exchange patient study files, presets, system configurations and request printing of reports, as will be described in more detail below. Third party machines with CypressLink capability can also be recognized. Units can identify themselves as any combination of Patient Database server (a device capable of storing data), Image Creation server (a device used for imaging and creating image data) or DICOM server, i.e. a server compatible with the DICOM imaging data format. It will be appreciated that other types of imaging file formats and networks may also be used with the disclosed architecture. For example, in addition to LAN's, other network technologies, such as wide are networks (“WAN”), intranets, extranet, the Internet, wireless networks, and combinations thereof, may also be used. It will be appreciated that the networking capabilities of the devices, as well as the type of networking technology used, are mutually interdependent and depend upon the particular implementation, and any such implementations may be used with the disclosed architecture.

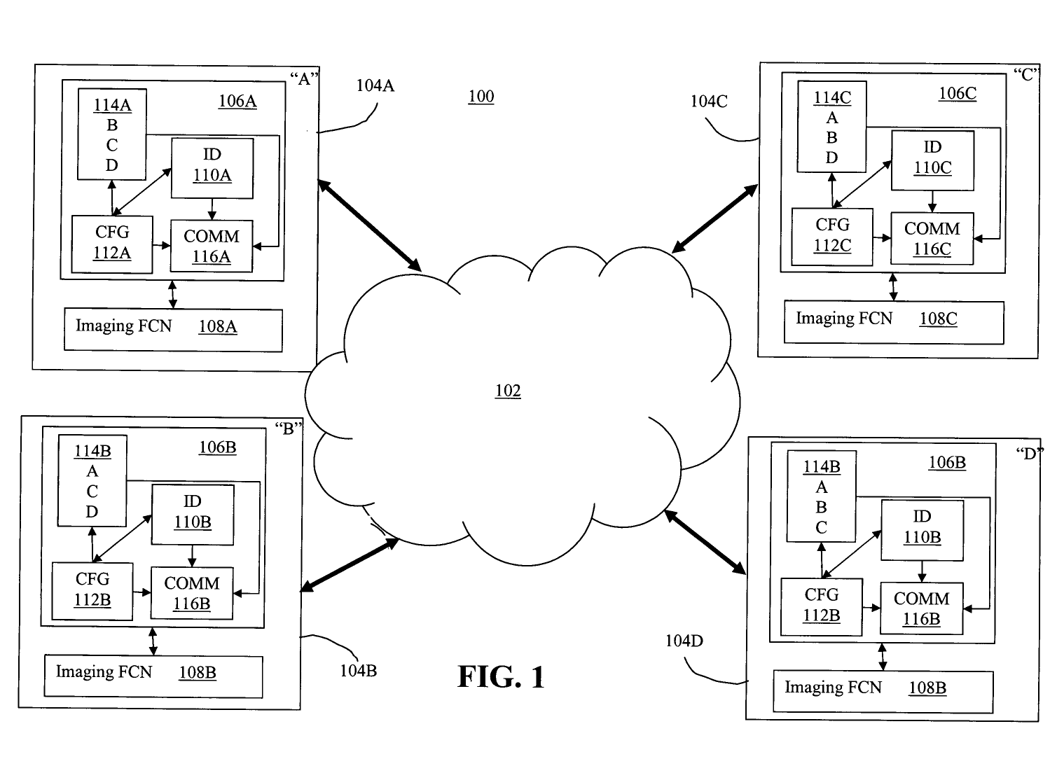

FIG. 1 shows an exemplary network 100 of diagnostic medical imaging devices 104A, 104B, 104C, 104D according to one embodiment. The devices 104A, 104B, 104C, 104D include diagnostic medical imaging systems, such as diagnostic medical ultrasound systems, MRI systems, etc. and/or diagnostic medical review stations. Each of the devices 104A, 104B, 104C, 104D is interconnected via a communications network 102, such as a LAN, WAN, wireless network or combinations thereof. Each device 104A, 104B, 104C, 104D includes a communications interface 106A, 106B, 106C, 106D and imaging functionality 108A, 108B, 108C, 108D. The communications interface 106A, 106B, 106C, 106D facilitates intra-device communications via the network 102 as will be described below. The imaging functionality 108A, 108B, 108C, 108D implements the various diagnostic medical imaging functions of the device 104A, 104B, 104C, 104D.

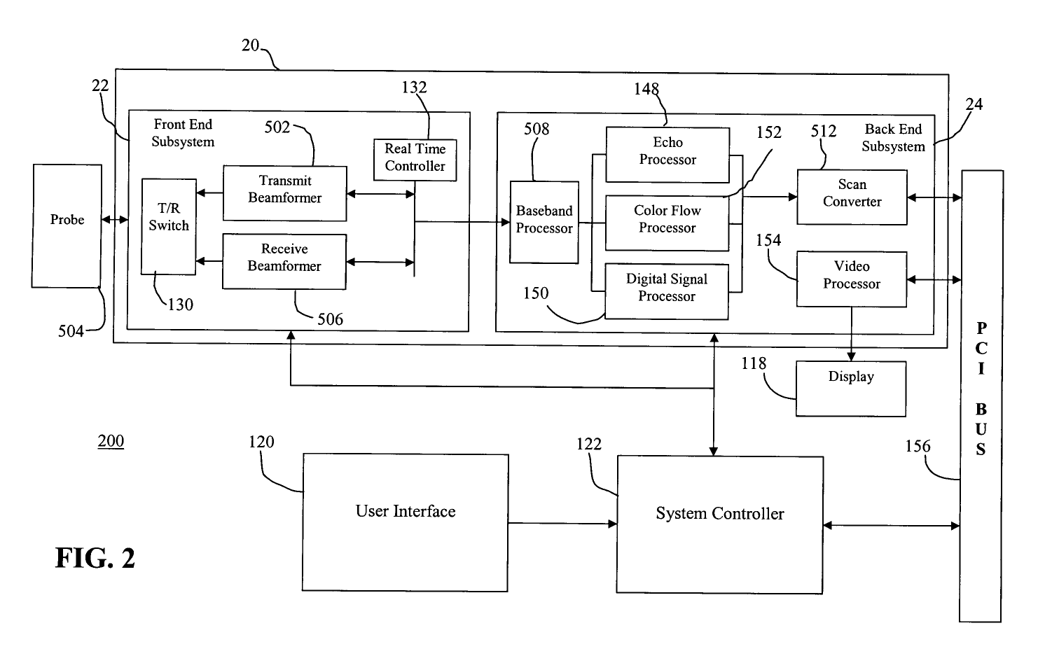

FIG. 2 shows one example of the imaging functionality 108A, 108B, 108C, 108D of a diagnostic medical ultrasound system 200 (104A, 104B, 104C, 104D), such as the Cypress Echocardiography System or the Sonoline Antares™ Ultrasound Platform, both manufactured by Siemens Medical Solutions USA, Inc., located in Malvern, Pa. The depicted ultrasound system architecture corresponds to the architecture of the Sonoline Antares™ Ultrasound Platform. It will be appreciated that one or more of the described components may be implemented in hardware, software or a combination thereof The ultrasound system 200 includes an ultrasonic imaging probe or transducer 504, acquisition hardware 20, a front end acquisition hardware subsystem 22, a back end acquisition hardware subsystem 24, a user interface 120, a system controller 122 and a display 118. In one embodiment, the back end subsystem 24 comprises a baseband processor 508, an echo processor 148, a color flow processor 152, a digital signal processor 150, a scan converter 512 and a video processor 154. In one embodiment, the exemplary front end acquisition hardware 22 includes a transmit beamformer 502, a receive beamformer 506, a transmit/receive switch 130, and a real time controller 132. As will be discussed below, the front end acquisition hardware 22 may alternatively include local or remote optical or magnetic data storage devices such as a computer memory, hard disk, CD, DVD or video tape recorder coupled with the ultrasound system 200 via a wired or wireless device or network interface. Herein, the phrase “coupled with” is defined to mean directly connected to or indirectly connected through one or more intermediate components. Such intermediate components may include both hardware and software based components.

The front end acquisition hardware 22 is coupled with the transducer 504. The front-end acquisition hardware 22 causes the transducer 504 to generate acoustic energy into a subject and receives the electrical signals generated by the transducer 504 in response to the received echoes representing a two dimensional representation of the subject. In one embodiment, the front end acquisition hardware 22 is configurable to acquire information corresponding to a plurality of two-dimensional representations or image planes of a subject for three-dimensional reconstruction. Other configurations, such as those for acquiring data with a two dimensional, 1.5 dimensional or single element transducer array, may be used. To generate each of the plurality of two-dimensional representations of the subject during an imaging session, the acquisition hardware 20 is configured to transmit, receive and process during a plurality of transmit events. Each transmit event corresponds to firing acoustic energy along one or more ultrasound scan lines in the subject. As a result of the succession of transmit events occurring during use of the system 500, information is received continuously throughout this process.

The transmit beamformer 502 is coupled with the transducer 504 and is of a construction known in the art, such as a digital or analog based beamformer capable of generating signals at different frequencies. The transmit beamformer 502 generates one or more excitation signals which causes the transducer 504 to emit one or more ultrasonic pulses. Each excitation signal has an associated center frequency. As used herein, the center frequency represents the frequency in a band of frequencies approximately corresponding to the center of the amplitude distribution. Preferably, the center frequency of the excitation signals is within the 1 to 15 MHz range and accounts for the frequency response of the transducer 504. The excitation signals have non-zero bandwidth.

It will be appreciated that alternative methods of generating and controlling ultrasonic energy as well as receiving and interpreting echoes received therefrom for the purpose of diagnostic imaging, now or later developed, may also be used with the disclosed embodiments in addition to or in substitution of current beamforming technologies. Such technologies include technologies which use transmitters and/or receivers which eliminate the need to transmit ultrasonic energy into the subject along focused beam lines, thereby eliminating the need for a transmit beamformer, and may permit beam forming to be performed by post processing the received echoes. Such post-processing may be performed by a receive beamformer or by digital or analog signal processing techniques performed on the received echo data. For example, please refer to U.S. patent application Ser. No. 09/518,972, entitled “METHOD AND APPARATUS FOR FORMING MEDICAL ULTRASOUND IMAGES”, now U.S. Pat. No. 6,309,356 and U.S. patent application Ser. No. 09/839,890, entitled “METHOD AND APPARATUS FOR FORMING MEDICAL ULTRASOUND IMAGES”, the disclosures of which are herein incorporated by reference.

Control signals are provided to the transmit beamformer 502 and the receive beamformer 506 by the real time controller 132. The transducer 504, as controlled by the transmit beamformer 502, is caused to fire one or more acoustic lines in each transmit event, and the receive beamformer 506 is caused to generate in-phase and quadrature (I and Q) information along one or more scan lines. Alternatively, real value signals may be generated. A complete frame of information corresponding to a two-dimensional representation (a plurality of scan lines) is preferably acquired before information for the next frame is acquired. The real time controller 132 is also used to manage the data flow created by the receive beamformer as it collects image information, making the stream of data available to the back end subsystem 22.

Upon the firing of one or more ultrasound scan lines into the subject, some of the acoustical energy is reflected back to the transducer 504. This reflected acoustical energy is detected by the transducer 504 and converted into electrical signals which are passed to the receive beamformer 506. In addition to receiving signals at the fundamental frequency (i.e., the same frequency as that transmitted), the non-linear characteristics of tissue or optional contrast agents also produce responses at harmonic frequencies. Harmonic frequencies are frequencies associated with non-linear propagation or scattering of transmit signals. As used herein, harmonic includes subharmonics and fractional harmonics as well as second, third, fourth, and other higher harmonics. Fundamental frequencies are frequencies corresponding to linear propagation and scattering of the transmit signals of the first harmonic. Non-linear propagation or scattering corresponds to shifting energy associated with a frequency or frequencies to another frequency or frequencies. The harmonic frequency band may overlap the fundamental frequency band.

The baseband processor 508 is coupled with the receive beamformer 506 and receives the converted electrical signals representative of the reflected acoustical energy. The baseband processor 108 passes information associated with a desired frequency band, such as the fundamental band or a harmonic frequency band. In one embodiment, the baseband processor 108 may be included as part of the receive beamformer 506. Furthermore, the baseband processor 108 demodulates the summed signals to baseband. The demodulation frequency is selected in response to the fundamental center frequency or another frequency, such as a second harmonic center frequency. For example, the transmitted ultrasonic waveforms are transmitted at a 2 MHz center frequency. The summed signals are then demodulated by shifting by either the fundamental 2 MHz or the second harmonic 4 MHz center frequencies to baseband (the demodulation frequency). Other center frequencies may be used. Signals associated with frequencies other than near baseband are removed by low pass filtering. As an alternative or in addition to demodulation, the baseband processor 108 provides band pass filtering. The signals are demodulated to an intermediate frequency (IF) (e.g. 2 MHz) or not demodulated and a band pass filter is used. Thus, signals associated with frequencies other than a range of frequencies centered around the desired frequency or an intermediate frequency (IF) are filtered from the summed signals. The demodulated or filtered signal is passed to the additional processors 148, 152 and 150 as either the complex I and Q signal or other types of signals, such as real value signals. It should be noted that band pass “filtering”, as well as other types of data filtering known in the art, should not be confused with the filter elements of the pipes and filters framework disclosed herein. As known in the art, “filtering” data involves allowing data with certain characteristics to pass while blocking data without those characteristics. On the other hand, while the filter elements discussed below may perform functions similar to those provided by the band pass processor 508, the filter elements, as used by the architecture described herein, are more general processing stages that manipulate, transform or enrich streaming data.

By selectively filtering which frequencies are received and processed, the backend subsystem 22 produces images with varying characteristics. In tissue harmonic imaging, no additional contrast agent is added to the target, and only the nonlinear characteristics of the tissue are relied on to create the ultrasonic image. Medical ultrasound imaging is typically conducted in a discrete imaging session for a given subject at a given time. For example, an imaging session can be limited to an ultrasound patient examination of a specific tissue of interest over a period of ¼ to 1 hour, though other durations are possible.

Tissue harmonic images provide a particularly high spatial resolution and often possess improved contrast resolution characteristics. In particular, there is often less clutter in the near field. Additionally, because the transmit beam is generated using the fundamental frequency, the transmit beam profile is less distorted by a specific level of tissue-related phase aberration than a profile of a transmit beam formed using signals transmitted directly at the second harmonic.

The harmonic imaging technique described above can be used for both tissue and contrast agent harmonic imaging. In contrast agent harmonic imaging, any one of a number of well known nonlinear ultrasound contrast agents, such as micro-spheres or the Optison™ agent by Nycomed-Amersham of Norway, are added to the target or subject in order to enhance the non-linear response of the tissue or fluid. The contrast agents radiate ultrasonic energy at harmonics of an insonifying energy at fundamental frequencies.

The echo 148, color flow 152 and digital signal 150 processors are coupled with the baseband processor 508 and receive the filtered signals from the transducer 504/receive beamformer 506. The digital signal processor 150 comprises one or more processors for generating two-dimensional Doppler or B-mode information. For example, a B-mode image, a color Doppler velocity image (CDV), a color Doppler energy image (CDE), a Doppler Tissue image (DTI), a Color Doppler Variance image, or combinations thereof may be selected by a user. The digital signal processor 150 detects the appropriate information for the selected image. In one embodiment, the digital signal processor 150 is adapted for Doppler processing and a B-mode processing. As known in the art, the Doppler processing estimates velocity, variance of velocity and energy from the I and Q signals. As known in the art, the B-mode processing generates information representing the intensity of the echo signal associated with the I and Q signals. The echo processor 148 performs baseband and amplitude mode signal processing of RF and IQ data in a known manner. The color flow processor 152 adds color to the acquired information, as known in the art.

The information generated by the echo 148, color flow 152 and digital signal 150 processors is provided to the scan converter 512. Alternatively, the scan converter 512 includes detection processes as known in the art and described in U.S. Pat. No. 5,793,701 entitled “METHOD AND APPARATUS FOR COHERENT IMAGE FORMATION”, assigned to the assignee of the present invention, the disclosure of which is herein incorporated by reference. The scan converter 512 is of a construction known in the art for arranging the output of the signal processors 148, 152 and 150 into two-dimensional representations or frames of image data. The scan converter 512 converts acoustic ultrasound line data, typically in a polar coordinate system, into data which may be plotted on a Cartesian grid. Using volume averaging or other similar algorithms on the returned echo data, the slice information is merged into a single 2D plane. This permits display of the ultrasound image on a two-dimensional output device such as a display monitor 118. Preferably, the scan converter 512 outputs formatted video image data frames, using a format such as the Cypress format, described above, the DICOM Medical industry image standard format or a TIFF format, or other image format presently known or later developed. Thus, the plurality of two-dimensional representations is generated. Each of the representations corresponds to a receive center frequency, such as a second harmonic center frequency, a type of imaging, such as B-mode, and positional information. It will be appreciated that the disclosed embodiments may also operate with ultrasound systems which produce three dimensional and/or four dimensional, i.e. real time 3-D, images. The harmonic based representations may have better resolution and less clutter than fundamental images. By suppressing the harmonic content of the excitation signal, the benefits of harmonic imaging of tissue may be increased. In any event, the scan converter 512 provides its output to the PCI bus 156. In one embodiment, the PCI bus 156 is a standard peripheral component interconnect board, as known, implemented in an IBM compatible personal computer system. An exemplary ultrasound system 200 implemented using an IBM compatible personal computer system having PCI bus, a Pentium class processor, manufactured by Intel Corporation, located in Santa Clara, Calif., and executing the Microsoft Windows XP Operating system, published by Microsoft Corporation, located in Redmond Wash., includes the Cypress Echocardiography System discussed above.

The user interface 120 is coupled with the system controller 122 and includes one or more input devices which the clinician/sonographer/physician uses to interface with the ultrasound system 200. The user interface 120 includes input devices such as a keyboard, mouse, trackball, touch screen or other input devices or combinations thereof as are known in the art. Further the user interface 120 may also include graphic user interface (“GUI”) elements coupled with the input devices and with the display 118 for both input and output functions. In addition to controlling the ultrasound functions of the ultrasound system 200, the user interface 120 may afford the user the opportunity to modify graphical representations, imaging planes and displays produced by the ultrasound system 200. Finally, the user interface 120 allows the user to coordinate multiple ultrasound probes 504.

The system controller 122 is coupled with the front end subsystem 22, the backend subsystem 22, the PCI bus 156 and the user interface 120 and controls and coordinates the functions of the ultrasound subsystems. The term “system controller” broadly refers to the appropriate hardware and/or software components of the ultrasound system 200 that can be used to implement the preferred embodiments described herein. It should be understood that any appropriate hardware (analog or digital) or software can be used and that the embodiments described herein can be implemented exclusively with hardware. Further, the system controller 122 can be separate from or combined with (in whole or in part) other processors of the ultrasound system 200 (including attendant processors), which are not shown in FIG. 2 for simplicity.

The various elements of the ultrasound system including the front end subsystem 22, backend subsystem 24 and user interface 120 are controlled in real time by the system controller 122. The system controller 122 controls the operation of the components of the system 200. A user, via the user interface 120, can adjust imaging parameters such as, but not limited to, image depth, image width, and frame rate. The controller 122 interprets the set-up information entered by the user and configures the components of the system 200 accordingly.

The video processor 154 acts as an interface between the system controller 122 and the display 118. In various embodiments, the video processor 154 can be configured to work with a variety of display types, such as cathode ray tubes or liquid crystal displays. The video processor 154 can also be configured to output information to a printer, memory, storage device, such as a computer storage device or a video recorder, computer network or other means for communicating data representative of an ultrasonic echo known in the art. The display monitor 118 is connected to the display controller 116 and is a standard display monitor as known in the art. In alternate embodiments, the display 118 can be replaced with a printer, memory, storage device, or any other output device known in the art.

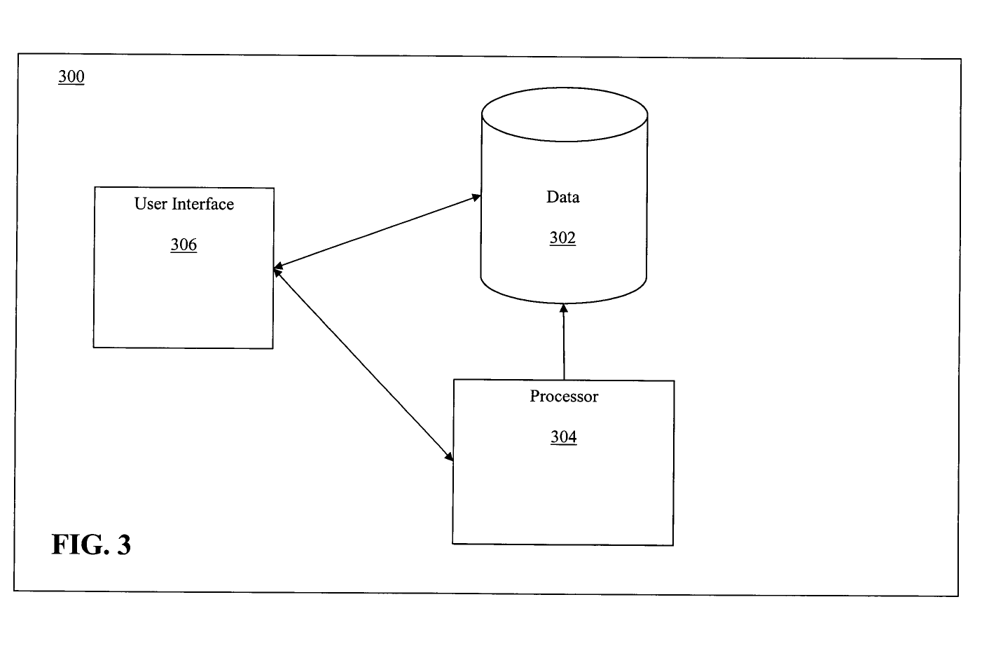

FIG. 3 shows an example of the imaging functionality 108A, 108B, 108C, 108D of a diagnostic medical imaging review station 300 (104A, 104B, 104C, 104D). The review station 300 includes a data storage device 302, a processor 304 and a user interface 306. The storage device 302 may include a computer memory, magnetic or optical storage device, or a combination thereof. The storage device 302 stores the operating software to operate the reviewing station and implement the reviewing functionality. The storage device 302 also stores the images and associated patient study files. The processor 304 executes the software and interfaces with the user via the user interface 306. The user interface 306 includes hardware, such as input and output devices, and software, such a as a graphic user interface, to interface the reviewing station functionality with the user of the reviewing station 300. In one embodiment, the user interface 306 includes a windows based graphic user interface which displays output to a user via a color display device, such as a CRT monitor or LCD flat panel display, and receives input via a keyboard, pointing device, such as a mouse, trackball or touch pad, or touch sensitive screen, or combinations thereof. An exemplary reviewing station 300 may include a personal computer workstation having a Pentium class processor, manufactured by Intel Corporation, located in Santa Clara, Calif., and executing the Cypress Viewer application, manufactured by Siemens Medical Solutions USA, Inc., located in Malvern, Pa. The exemplary station 300 further includes a color display, 512 Megabytes of RAM, a 20 Gigabyte, or larger, fixed hard disk and an optical drive, such as a CD-ROM drive, and executes the Windows XP operating system, manufactured by Microsoft Corporation, located in Redmond, Wash. It will be appreciated that the review station 300 can include any suitable general purpose or special purpose computer, capable of implementing the disclosed functionality.

It will be appreciated that other devices 104A, 104B, 104C, 104D may also be coupled with the network 102 having other imaging functionality 108A, 108B, 108C, 108D. Such other imaging functionality 108A, 108B, 108C, 108D may include image storage or image output functionality.

Referring back to FIG. 1, each device 104A, 104B, 104C, 104D further includes an communications interface 106A, 106B, 106C, 106D which couples the device 104A, 104B, 104C, 104D with the network 102. The communications interface 106A, 106B, 106C, 106D includes identification logic 110A, 110B, 110C, 110D, configuration logic 112A, 112B, 112C, 112D and communications logic 116A, 116B, 116C, 116D. The communications interface 106A, 106B, 106C, 106D further includes appropriate interfaces (not shown) with the network 102 and the imaging functionality 108A, 108B, 108C, 108D which are dependent upon the implementation of the device 104A, 104B, 104C, 104D, such as the type of network 102 or the type of operating system executing on the device 104A, 104B, 104C, 104D. In one embodiment, the network 102 comprises a Transport Control Protocol/Internet Protocol (“TCP/IP”) based network, either wired, wireless or a combination thereof, and the communications interface 106A, 106B, 106C, 106D includes the appropriate hardware and software, such as a TCP/IP protocol stack, wireless network adapter or Ethernet adapter, for communications over such a network 102.

As will be described below, the identification logic 110A, 110B, 110C, 110D continually operates to identify other devices 104A 104B, 104C, 104D on the network 102 and respond to the identification communications of other devices 104A 104B, 104C, 104D. In one embodiment, the identification logic 110A, 110B, 110C, 110D utilizes the TCP/IP multicast protocol to broadcast an identification message out to all devices on the network 102, listens for responses and engages in an exchange of communications to establish communications. The configuration logic 112A, 112B, 112C, 112D is coupled with the identification logic 110A, 110B, 110C, 110D and configures the device 104A, 104B, 104C, 104D based on the identification of other devices 104A, 104B, 104C, 104D on the network 102 identified as being available for communications by the identification logic 110A, 110B, 110C, 110D. In one embodiment, as each available device 104A, 104B, 104C, 104D is identified, the configuration logic 112A, 112B, 112C, 112D adds a representation of the identified device 104A, 104B, 104C, 104D, such as a network name, alias or address, to a list 114A, 114B, 114C, 114D of available devices. This list 114A, 114B, 114C, 114D is made available to the user of the device 104A, 104B, 104C, 104D to select from when choosing to establish communications from one device 104A, 104B, 104C, 104D to another device 104A, 104B, 104C, 104D. Once a user has selected another device 104A, 104B, 104C, 104D to communicate with from the list of available devices 114A, 114B, 114C, 114D, the communications logic 116A, 116B, 116C, 116D facilitates the exchange of information between the devices 104A, 104B, 104C, 104D, such as by transferring the requested data file in the requested manner, as will be described below.

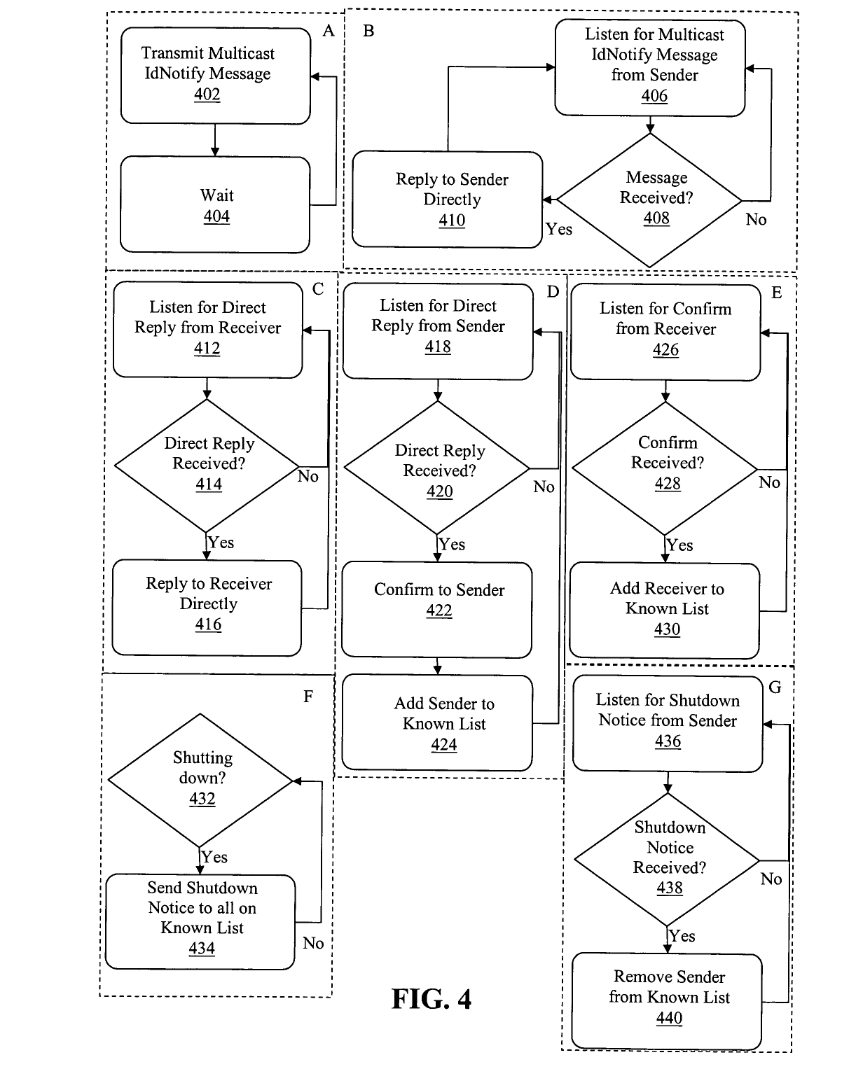

FIG. 4 shows flow charts A-G of the processes executed by the identification logic 110A, 110B, 110C, 110D and configuration logic 112A, 112B, 112C, 112D. Each of these processes A-G are executed substantially concurrently to implement the automated discovery, configuration and maintenance functionality of the disclosed architecture.

In process A, the identification logic 110A, 110B, 110C, 110D of each device 104A, 104B, 104C, 104D (the “multicast device”) transmits a multicast identification message (block 402), referred to as an “IdNotify” message, and then waits for a preset period of time (block 404), such as 30 seconds, before transmitting the multicast identification message again. Each device 104A, 104B, 104C, 104D may begin multicasting identification messages as soon as it is powered on and ready or each device 104A, 104B, 104C, 104D may wait for a synchronizing event, such as a signal transmitted over the network, to begin multicasting. Multicast is a facility of the TCP/IP protocol that permits sending network packets to multiple devices on a network simultaneously. Multicast is similar to broadcasting, only more efficient in that only machines that have requested receiving packets sent to a particular IP address, receive those packets. The identification message includes information such as the device 104A, 104B, 104C, 104D name, the Internet Protocol (“IP”) address of the device 104A, 104B, 104C, 104D, as well as other communications related parameters, such as the type of device 104A, 104B, 104C, 104D or its capabilities, such as whether it is a device capable of creating image data or capable of storing data. In one embodiment, multicast aware network routers may be used to separate network segments. In such an embodiment, the Time To Live (“TTL”) parameter of the TCP/IP packet can be used to control the “network distance”, i.e. the number of routers, that the multicast messages can reach.

In one embodiment of the disclosed architecture, all communications, including the identification messages, include one or more messages (packets) from a source to a destination device 104A, 104B, 104C, 104D and one or more messages in reply. Messages consist of a null-terminated string of ANSI characters, followed in some cases by a block of binary file content data. The character string portion of the message is a set of tagged (or name-value pair) items. An ‘=’ character separates the tag (name) from the value. A semi-colon separates each tagged item from the next. No whitespace is used around the ‘=’ or ‘;’, thus names and values may contain whitespace.

In this embodiment, tags consist of one of the following set of defined data items used to make up messages:

TABLE 1

| Tag | Meaning |
|---|---|
| Scope | Message is being multi cast or directed to |
| | one machine. |
| Type | Message is a machine ID, status reply, or |
| | part of a file transfer etc. |
| DeviceName | Name of the machine that is represented by |
| | an ID message. |
| DeviceType | Type (Imaging or Review) of the machine |
| | that is represented by an ID message. |
| DicomAeTitle | DICOM server parameter. |
| DicomPortNumber | DICOM server parameter. |
| DicomServer | Boolean - value == “Yes” or “No”. |
| PatientDataServer | Boolean - value == “Yes” or “No”. |
| ImageCreator | Boolean - value == “Yes” or “No”. |
| TimeToNext | Time in milliseconds until sender sends |
| | again. |
| FileName | Name of file to be transferred. |
| FileDataSize | Size in bytes of file. |
| Status | Indicate in a reply the success or reason for |
| | failure. |

All message have a Scope and a Type item. Other items only exist as appropriate per the value of the Type item. Version issues will be avoided by ensuring unrecognized tags are ignored. New versions of software can then supply appropriate default values for missing fields. In an alternate embodiment, the existing “comment” field beside a workstation's name will be used to allow identifying a machine in a more user friendly way. It will not have any special meaning to the software. A new Tag will be defined for it. It will be appreciated that other message formats may be used with the disclosed embodiments, and that such formats are implementation dependent.

In process B, the identification logic 110A, 110B, 110C, 110D listens for the multicast identification message from other multicast devices 104A, 104B, 104C, 104D on the network 102 (block 406). For the purposes of this discussion, the device 104A, 104B, 104C, 104D that sends the multicast identification message will be referred to as the “sending device” and the device 104A, 104B, 104C, 104D which receives that multicast identification message will be referred to as the “receiving device.” It will be appreciated that at any given time, each machine is both a sending and receiving device as the transmission and reception of multicast identification messages occurs substantially simultaneously, as was described above. As will be described, each received multicast message causes the multicast device 104A, 104B, 104C, 104D to be added to a list 114A, 114B, 114C, 114D of known remote machines that are available as candidates for the exchange of data. In one embodiment, devices 104A, 104B, 104C, 104D that are to recognize each other must use the same multicast IP address. This permits one network 102 to support independent groups of devices 104A, 104B, 104C, 104D that communicate among each other, but not between the independent groups, by using separate multicast addresses. Once an identification message is received (block 408) the receiving device 104A, 104B, 104C, 104D transmits a reply message directly back to the sending device 104A, 104B, 104C, 104D (block 410).

This reply sequence, plus the interactive confirmation sequence described below, allows a device 104A, 104B, 104C, 104D to learn of another device either by receiving a multicast identification message or receiving a reply to their multicast identification message. This ensures that devices 104A, 104B, 104C, 104D that come on the network later, find out about the presence of machines already on the network, without waiting for the multicast identification interval, i.e. by sending their own identification message and not having to wait to receive identification messages from other devices 104A, 104B, 104C, 104D.

In one embodiment, all replies in the sequence are standard UDP/IP messages, directed to the specific IP address of the intended machine. The overall purpose of the sequence is to ensure that when, for instance, device A puts device B in its table of known devices, it has completed both a directed send and a directed receive with that device 104A, 104B, 104C, 104D. This ensures that direct communications are functioning properly as sometimes multicast communications work even when direct communications fail.

As will be described, each device will respond to a multicast by sending a reply to the multicaster. A reply is directed back to the IP address that was received in the multicast. The format of the reply is the same as the multicast, except for the Scope item. The Scope item will have the value “Direct”. The machine receiving the reply with a scope “Direct” responds with a reply with scope “DReply”. When the original multicasting machine gets this “DReply” it puts the sending machine in its table of known machines and send a reply with a scope “DCnfrm”. When the “DCnfrm” reply is received by the second machine, it places the first machine in its table of known machines.

In process C, the identification logic 110A, 110B, 110C, 110D listens for a direct reply from a receiving device 104A, 104B, 104C, 104D of its multicast identification message (block 412). If such a reply is received, a direct reply message is generated back to that receiving device 104A, 104B, 104C, 104D (block 416).

In process D, the identification logic 110A, 110B, 110C, 110D listens for direct replies from the sending device 104A, 104B, 104C, 104D in response to direct replies from the receiving device 104A, 104B, 104C, 104D generated in response to the multicast identification message (block 418). If such a direct reply is received (block 420), the receiving device 104A, 104B, 104C, 104D generates a confirmation message back to the sending device 104A, 104B, 104C, 104D (block 422) and causes the configuration logic 112A, 112B, 112C, 112D to add the sending device to its list 114A, 114B, 114C, 114D of devices 104A, 104B, 104C, 104D available for communication (block 424).

In process E, the identification logic 110A, 110B, 110C, 110D listens for confirmation messages from receiving devices 104A, 104B, 104C, 104D (block 426). Upon receipt of a confirmation message from a receiving device 104A, 104B, 104C, 104D (block 428), the identification logic 110A, 110B, 110C, 110D of the sending device 104A, 104B, 104C, 104D causes the configuration logic 112A, 112B, 112C, 112D to add the receiving device 104A, 104B, 104C, 104D to its list 114A, 114B, 114C, 114D of devices 104A, 104B, 104C, 104D available for communication (block 430).

In one embodiment, each device 104A, 104B, 104C, 104D may maintain up to 256 devices on its list 114A, 114B, 114C, 114D of devices 104A, 104B, 104C, 104D available for communication. Where more than 256 devices 104A, 104B, 104C, 104D exist on the network 102, only the first 256 devices 104A, 104B, 104C, 104D to respond to the multicast identification message will be listed. It will be appreciated that the number of devices 104A, 104B, 104C, 104D that can be listed is implementation dependent.

In this way, each device 104A, 104B, 104C, 104D on the network 102 discovers the other devices 104A, 104B, 104C, 104D and configures itself for communications. Further, as the multicast identification messages are periodically repeated, changes in network topology or available devices 104A, 104B, 104C, 104D are picked up by the other devices 104A, 104B, 104C, 104D.

In one embodiment, to ensure that failing devices 104A, 104B, 104C, 104D are removed from the remaining devices' 104A, 104B, 104C, 104D lists 114A, 114B, 114C, 114D of devices 104A, 104B, 104C, 104D available for communication, a device 104A, 104B, 104C, 104D is removed if there has been no reply received from that device 104A, 104B, 104C, 104D after a fixed number of multicast cycles. In one embodiment, devices 104A, 104B, 104C, 104D are removed after two cycles without a reply. Therefore, active devices 104A, 104B, 104C, 104D must respond to multicast identification messages to remain on the lists 114A, 114B, 114C, 114D of devices 104A, 104B, 104C, 104D available for communication.

In one embodiment, the identification logic further provides processes to allow devices that are being shut down to remove themselves from the other devices' 104A, 104B, 104C, 104D lists 114A, 114B, 114C, 114D of devices 104A, 104B, 104C, 104D available for communication. In process F, when a device 104A, 104B, 104C, 104D is shutting down (block 432), its identification logic 110A, 110B, 110C, 110D transmits a shutdown message to all of the devices 104A, 104B, 104C, 104D on its list 114A, 114B, 114C, 114D of devices 104A, 104B, 104C, 104D available for communication (block 434). In process G, the identification logic 110A, 110B, 110C, 110D listens for shutdown messages (block 436). Upon receipt of a shutdown message (block 438), the identification logic 110A, 110B, 110C, 110D causes the configuration logic 112A, 112B, 112C, 112D to remove the shutdown device 104A, 104B, 104C, 104D from its list 114A, 114B, 114C, 114D of devices 104A, 104B, 104C, 104D available for communication (block 440).

The discovery process is summarized in the following table:

TABLE 2

| Device A | Device B |
|---|---|
| Multicasts an IdNotify message to all | |
| listening on the multicast address. | |
| | Receives A's multicast IdNotify, |
| | replies with its own IdNotify, with |
| | scope Direct, only to A. |
| Receives B's Direct IdNotify, replies | |
| with its own IdNotify, with scope | |
| DReply, only to B. | |
| | Receives A's DReply IdNotify, |
| | replies with its own IdNotify, with |
| | scope DCnfrm, only to A. Places A |
| | in its table of known machines. |
| Receives B's DCnfrm IdNotify. | |
| Places B in its table of known | |
| machines. | |

In an alternate embodiment, devices 104A, 104B, 104C, 104D include wireless devices and network 102 includes a wireless network, such as a network compatible with the 801.11b wireless network protocol, or combination of wired and wireless network. In this embodiment, the wireless devices may further issue shutdown messages, as described above, when they detect that they leaving the range of the wireless network 102. When re-entering the range of the wireless network 102, the wireless devices may resume normal participation in the multicast protocol described above.

As described above, each device 104A, 104B, 104C, 104D builds a list 114A, 114B, 114C, 114D of other devices 104A, 104B, 104C, 104D that can be communicated with. This list 114A, 114B, 114C, 114D is made available to the user of the device 104A, 104B, 104C, 104D to allow the user to initiate communications over the network 102 via the communications logic 116A, 116B, 116C, 116D. In one embodiment, the communications logic 116A, 116B, 116C, 116D supports “push” file transfers and “pull” file transfers. Push file transfers allow the user of the device 104A, 104B, 104C, 104D to send data (“upload”) to another device 104A, 104B, 104C, 104D. Pull file transfers allow the user of the device 104A, 104B, 104C, 104D to retrieve data (“download”) from another device 104A, 104B, 104C, 104D.

The data that can be communicated between devices 104A, 104B, 104C, 104D includes patient study files, including images, machine configuration files and other data or system files.

Essentially, to transfer a file from the user's device 104A, 104B, 104C, 104D to another device 104A, 104B, 104C, 104D on the network 102, the user uses the user interface of their device 104A, 104B, 104C, 104D to select the destination device 104A, 104B, 104C, 104D from the list 114A, 114B, 114C, 114D of other devices 104A, 104B, 104C, 104D that can be communicated with. The user then selects the file to be transferred and the communication logic 116A, 116B, 116C, 116D transfers the file as will be described below.

To retrieve a file from another device 104A, 104B, 104C, 104D on the network 102, the user uses the user interface of their device 104A, 104B, 104C, 104D to select the source device 104A, 104B, 104C, 104D from the list 114A, 114B, 114C, 114D of other devices 104A, 104B, 104C, 104D that can be communicated with. The user then indicates that he wishes to retrieve a file which causes a list of available data files to be retrieved from the source device 104A, 104B, 104C, 104D. The user then selects the file to be transferred and the communication logic 116A, 116B, 116C, 116D transfers the file as will be described below. In one embodiment, only devices 104A, 104B, 104C, 104D which identify themselves as “patient database servers” or otherwise as a device accessible for file retrieval, will be available to retrieve files from. In this way, devices 104A, 104B, 104C, 104D can control whether they are available for push or pull type file transfers.

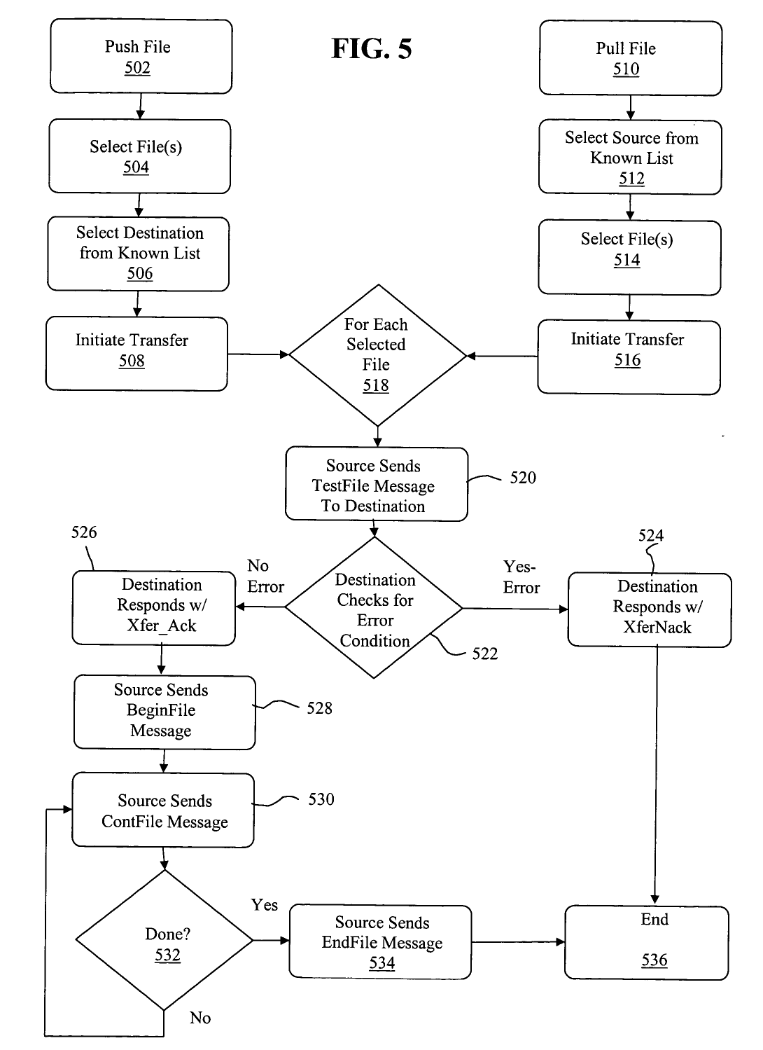

FIG. 5 shows a flow chart depicting the process of transferring files using the disclosed architecture. The depicted processes can be invoked as long as there is at least one other device 104A, 104B, 104C, 104D available on the network 102 for communications and the protocols described above have been executed. The user first determines whether they want to send a file from their device 104A, 104B, 104C, 104D to another device 104A, 104B, 104C, 104D on the network 102 or retrieve a file from another device 104A, 104B, 104C, 104D (blocks 502, 510). If the user elects to send a file, they then select the file or files to be transferred (block 504) and select the destination device 104A, 104B, 104C, 104D (block 506) as was described above. If the user elects to retrieve a file, they first select the source device 104A, 104B, 104C, 104D, as described above (block 512) and then select the file or files from the list of available files retrieved from the source device 104A, 104B, 104C, 104D (block 514).

In one embodiment, each file is transferred one at a time. The user may request more than one file in a batch and each will be sent in an individual request/reply transaction, as described below. The transactions will be performed serially. It will be appreciated, that where the networking protocols permit, parallel transactions may be implemented to allow substantially simultaneous transfer of multiple files.

In an alternate embodiment, a user may cause a file to be transferred to multiple destination devices 104A, 104B, 104C, 104D substantially simultaneously.

For each selected file (block 518), the source device 104A, 104B, 104C, 104D sends a TestFile message to the destination device 104A, 104B, 104C, 104D, which includes the filename (block 520). The destination device 104A, 104B, 104C, 104D then checks for error conditions (block 522) such as whether there is space to store the file. In an alternate embodiment, the source device 104A, 104B, 104C, 104D also checks for error conditions, such as whether the file to be transferred exists or not, is locked or otherwise protected or in use. The destination device 104A, 104B, 104C, 104D will reply with an Xfer_Ack or XferNack message (block 526, 524). An XferNack is sent when an error condition occurs, such as a duplicate filename, full directory, or platform/version incompatibility etc. An Xfer_Ack reply indicates ready for file transfer. In an XferNack is received, the file transfer process terminates and an error is returned to the user via the user interface (block 536).

The source device 104A, 104B, 104C, 104D then sends a BegnFile message (block 528). This includes up to the first 64 KB of the file data. This followed by as many ContFile messages as necessary (block 530, 532), each containing 64 KB of file data. Finally an End_File is sent with the last block of file data (block 534). If the entire file fit in the first BegnFile (less than 64K), an End_File with no data will be sent to terminate the file transfer.

The file will be stored as a new file on the receiving device 104A, 104B, 104C, 104D, with the same filename as the source machine. In one embodiment, a Globally Unique Identifier (“GUID”) is associated with the file and used to uniquely identify it. A GUID is a 128 bit number provided by the Microsoft Windows operating system that can be used for any application-specific purpose, and once assigned, is guaranteed to be unique across all computers at all times. It will be appreciated that any unique identifier, such as the filename or GUID may be used to identify data files for purposes of the disclosed embodiments. It is stored in the InBox database directory for that device 104A, 104B, 104C, 104D. In one embodiment, only the permanent data in the image study file will be sent, not the XY data. Files may be compacted or otherwise compressed before sending.

In one embodiment of the disclosed architecture, the program code for implementing the disclosed processes and functionality is included within a Dynamic Link Library (“DLL”) file and is incorporated into the appropriate application via linking. For a review station device 104A, 104B, 104C, 104D, a separate application is wrapped around the DLL. This allows the application to run all the time on the review station device 104A, 104B, 104C, 104D and receive studies, even when the review station application is not running. In this embodiment, the application executes as an NT service which places an icon on the display. Selecting this icon will display a user interface that can be used to see the list of machines discovered on the network and perform other diagnostic operations.

In an alternate embodiment, the disclosed architecture may also be used to transfer data files between devices 104A, 104B, 104C, 104D which contain device 104A, 104B, 104C, 104D configuration settings, parameters or data. Such configuration data may include any information that the user may modify to adapt their machine to their liking, such as set up values, preset database files, vascular site database files, text or audio annotations. The variety and type of parameters available for transfer is dependent on the type of device 104A, 104B, 104C, 104D, e.g. a diagnostic medical ultrasound system may have many more configurable parameters than a viewing workstation.

The ability to transfer configuration data may be used for backing up such settings, such as by storing them on another device 104A, 104B, 104C, 104D for safe-keeping. The configuration data may then be retrieved at a later time to restore the settings. Configuration data may also be transferred for the purpose of duplicating settings. For example, an operator who uses more than one device 104A, 104B, 104C, 104D may want to have his custom configurations available on both devices. In one embodiment, the transfer of configuration data overwrites the current configuration of the destination device 104A, 104B, 104C, 104D. Alternatively, the configuration data may be merged, where appropriate, with the configuration data currently on the destination device 104A, 104B, 104C, 104D. Whether to overwrite or merge settings may be at the operator's discretion.

Transfer of configuration files is handled the same way as other data files, as described above. Where the configuration data is spread across multiple configuration files, the user may transfer all of the files with a single action.

As described above, an intra-device communications architecture is disclosed which facilitates automated discovery of devices 104A, 104B, 104C, 104D on a network 102 which are available for communications. Devices 104A, 104B, 104C, 104D communicate information about themselves over a network 102, so that all of them can create a list of others seen on the network 102 automatically. Thus there is no user data entry required for one device 104A, 104B, 104C, 104D to know about the existence of other devices 104A, 104B, 104C, 104D, and be able to send or retrieve files. Each device 104A, 104B, 104C, 104D sends its name so a user can recognize machines in a list of all device 104A, 104B, 104C, 104D on the network 102. Each device 104A, 104B, 104C, 104D sends the other devices 104A, 104B, 104C, 104D enough information (network address etc.) to allow for transfer of files back to it. The intra-device 104A, 104B, 104C, 104D communication repeats frequently enough to avoid stale entries in the list of devices 104A, 104B, 104C, 104D on the network 102. If a device 104A, 104B, 104C, 104D stops communicating on the network 102, other devices 104A, 104B, 104C, 104D are able to remove it from their lists 114A, 114B, 114C, 114D. Each device 104A, 104B, 104C, 104D includes the duration for which its identification data remains valid, along with the data. This allows destination device 104A, 104B, 104C, 104D to timeout the data, based on when the sender will send again. Any receipt of a packet of device 104A, 104B, 104C, 104D identification information from a remote device 104A, 104B, 104C, 104D, causes the receiving device 104A, 104B, 104C, 104D to send a identification packet back to the sender. This ensures that when a device 104A, 104B, 104C, 104D starts up on the network 102, it learns about existing devices 104A, 104B, 104C, 104D on the network as soon as possible without having to wait for the repeat interval. When a device 104A, 104B, 104C, 104D is shutdown it sends a packet notifying other devices 104A, 104B, 104C, 104D. This allows immediate removal from their lists 114A, 114B, 114C, 114D.

Once the devices 104A, 104B, 104C, 104D have identified each other, the user may transfer files among them, as described. The user interface for sending data, such as Study Files, allows the user to select a destination device 104A, 104B, 104C, 104D from the list 114A, 114B, 114C, 114D of devices 104A, 104B, 104C, 104D acquired from the network 102. A device 104A, 104B, 104C, 104D attempting to send data, such as a study file, must be able to verify that the destination device 104A, 104B, 104C, 104D can accept the data. This means that there is sufficient disk space for the file, it doesn't already exist and/or that some other device 104A, 104B, 104C, 104D is not trying to send data to the same destination at the same time with the same name. Devices 104A, 104B, 104C, 104D are able to send the data to the destination machine, once space etc. has been verified. All devices 104A, 104B, 104C, 104D are able to accept a file from any other device 104A, 104B, 104C, 104D and multiple devices 104A, 104B, 104C, 104D may be sending to the same destination simultaneously. File transfer operations are able to detect network errors and report any failure to complete the file transfer.

It is therefore intended that the foregoing detailed description be regarded as illustrative rather than limiting, and that it be understood that it is the following claims, including all equivalents, that are intended to define the spirit and scope of this invention.

1. A method for communicating among a plurality of diagnostic medical imaging devices coupled with a network, said method comprising:
identifying, automatically by a first device of said plurality of diagnostic medical imaging devices, a second device of said plurality of diagnostic medical imaging devices available for communication via said network;
configuring, automatically, said first device to communicate substantially directly with said second device via said network; and
facilitating substantially direct communication of data between said first and second devices.

2. The method of claim 1, wherein said identifying further comprises:
receiving, by said first device, a first identification message periodically transmitted by said second device to all of said plurality of diagnostic medical imaging devices;
transmitting a reply, by said first device, to said second device in response to said first identification message;
receiving, by said first device, a second identification message transmitted by said second device to said first device in response to said reply; and
transmitting a confirmation, by said first device, to said second device in response to said second identification message.

3. The method of claim 2, further comprising:
configuring said second device to communicate substantially directly with said first device in response to said confirmation.

4. The method of claim 1, wherein said configuring further comprises:
appending a representation of said second device to a list of representations of devices available for communication maintained on said first device.

5. The method of claim 1, wherein said facilitating further comprises:
receiving a request from a user of said first device to send data from said first device to said second device;
transmitting said data from said first device to said second device.

6. The method of claim 1, wherein said facilitating further comprises:
receiving a request from a user of said first device to send data from said second device to said first device;
transmitting a request for said data to said second device; and
receiving said data in response to said request.

7. A communications interface for a first diagnostic medical imaging device, said communications interface operative to couple said first diagnostic medical imaging device to a network, said communications interface comprising:
identification logic operative to periodically identify, via said network, said first diagnostic medical imaging device to other diagnostic medical imaging devices coupled with said network and receive a response therefrom, said identification logic being further operative to recognize other diagnostic medical imaging devices which identify themselves to said first diagnostic medical imaging device;
configuration logic coupled with said identification logic and operative to automatically configure said first diagnostic medical imaging device to communicate with said other diagnostic medical imaging devices which at least one of respond and identify themselves; and
communication logic coupled with said identification logic and said configuration logic and operative to facilitate communication of data between said first diagnostic medical imaging device and said other diagnostic medical imaging devices which at least one of respond and identify themselves.

8. The communications interface of claim 7, wherein said identification logic is further operative to periodically broadcast an identification message to said other diagnostic medical imaging devices, said identification message operative to solicit responses from said other diagnostic medical imaging devices, wherein upon receipt of a solicited response from a one of said other diagnostic medical imaging devices, said identification logic is further operative to transmit a confirmation request to said one of said other diagnostic medical imaging device, and wherein said configuration logic is further operative to configure said first diagnostic medical imaging device based on receipt of a response to said confirmation request.

9. The communications interface of claim 7, wherein said identification logic is further operative to receive an unsolicited identification message from one of said other diagnostic medical imaging devices, said identification logic being operative to transmit a reply message to a sender of said unsolicited identification message and transmit a confirmation message to said sender in response to receipt of a confirmation request.

10. The communications interface of claim 7, wherein said communication logic is further operative to receive a selection from a user of data and one of said other diagnostic medical imaging devices, said communication logic being operative to transmit said data from said first diagnostic medical imaging device to said one of said other diagnostic medical imaging devices.

11. The communications interface of claim 7, wherein said communications logic is further operative to receive a selection from a user of one of said other diagnostic medical imaging devices, said communications logic being further operative to request that said one of said other diagnostic medical imaging devices identify data stored therein in response to said selection, and wherein a representation of said identified data is provided to said user, said communication logic being further operative to receive a selection from said user of data from said identified data and, in response to said selection, transmit a request for said data to said one of said other diagnostic medical imaging device.

12. An communications architecture comprising:
a network;
a plurality of diagnostic medical imaging devices, each of said plurality of diagnostic medical imaging devices being coupled with said network;
each of said plurality of diagnostic medical imaging devices being operative to automatically discover at least one other of said plurality of diagnostic medical imaging devices via said network and facilitate communications therebetween.

13. The communications architecture of claim 12, wherein each of said plurality of diagnostic medical imaging devices further include a communications interface, said communications interface operative to couple said diagnostic medical imaging device with said network, said communications interface being further operative to:
transmit, periodically, an identification message to all other of said plurality of diagnostic medical imaging devices;
transmit a reply message in response to receipt of said identification message from another of said plurality of diagnostic medical imaging devices, said reply message being transmitted to a sender of said identification message;
transmit a confirmation request in response to receipt of a reply message from another of said plurality of diagnostic medical imaging devices, said confirmation request being transmitted to a sender of said reply message; and
transmit a confirmation in response to receipt of a confirmation request from another of said plurality of diagnostic medical imaging devices, said confirmation being transmitted to a sender of said confirmation request; and
enable communications with a sender of said confirmation request;
enable communications with a sender of said confirmation.

14. The communications architecture of claim 12, wherein said plurality of diagnostic medical imaging devices include at least one device selected from the group comprising: diagnostic a medical image acquisition system, a diagnostic medical imaging reviewing workstation, a diagnostic medical imaging server, and a diagnostic medical patient monitor.

15. The communications architecture of claim 12, wherein said network comprises at least one of a wired and wireless network.

16. An communications architecture comprising:
a plurality of diagnostic medical imaging devices;
networking means for interconnecting each of said plurality of diagnostic medical imaging devices; and
wherein each of said plurality of diagnostic medical imaging devices comprises means for automatically discovering at least one other of said plurality of diagnostic medical imaging devices via said network and facilitating communications therebetween.
